(19) United States
(12) Patent Application Publication
(10) Pub. No.: US 2026/0310854 A1
(43) Pub. Date: Oct. 8, 2026

(54) NUMERICAL SIMULATION METHOD AND DEVICE FOR TARGET TEMPERATURE RISE, STORAGE MEDIUM AND TERMINAL

(71) Applicant: Huapeng Neutron Technology (Hangzhou) Co., Ltd., Hangzhou (CN)

(72) Inventors: Sheng WANG, Hangzhou (CN); Yaocheng HU, Hangzhou (CN); Jinglun LI, Hangzhou (CN); Yupeng XIE, Hangzhou (CN)

(73) Assignee: Huapeng Neutron Technology (Hangzhou) Co., Ltd., Hangzhou (CN)

(21) Appl. No.: 19/256,205

(22) Filed: Jul. 1, 2025

(30) Foreign Application Priority Data
Jul. 3, 2024 (CN) 2024108833500

(51) Int. Cl. G06F 30/23; G06F 111/10

(52) U.S. Cl. CPC G06F 30/23; G06F 2111/10

(57) ABSTRACT

The present invention provides a numerical simulation method and device for target temperature rise, a storage medium and a terminal. The numerical simulation method comprises obtaining a target finite element model, obtaining a beam parameter of a charged particle beam; obtaining corresponding particle spatial position coordinate distribution data; performing coordinate fitting on a charged particle distribution in three dimensions to obtain an expression of the corresponding charged particle distribution along a preset two-dimensional direction as a charged particle distribution feature corresponding to the beam parameter; obtaining a simulation operation condition parameter, and simulating a process of the beam bombarding the target finite element model using finite element analysis based on the simulation operation condition parameter and the charged particle distribution feature to obtain a target surface temperature distribution after the target finite element model is bombarded. The present invention can effectively realize temperature evaluation of target temperature rise.

### FIELD OF THE INVENTION

The present invention belongs to the technical field of Boron Neutron Capture Therapy (BNCT), relates to a numerical simulation method for target temperature rise, and in particular to a numerical simulation method and device for target temperature rise caused by charged particle bombardment, a storage medium and a terminal.

### BACKGROUND OF THE INVENTION

BNCT is a cancer treatment that leverages neutron-boron interactions. FIG. 1 shows a schematic diagram of BNCT treatment. Referring to FIG. 1, this technology uses boron-10 isotopes, which capture thermal neutrons (low-energy neutrons) and undergo a nuclear reaction, producing high-energy α-charged particles and lithium-7 nuclei. The resulting energy is concentrated within cancer cells, destroying cancer cells while sparing surrounding healthy tissues, BNCT thus offers a targeted radiotherapy approach. The therapeutic neutrons are generated by bombarding a target (e.g., a lithium target) with charged particles (e.g., protons or heavy ions) accelerated by an accelerator. During the bombardment of the target by the charged particle beam, the energy deposition of the charged particles causes the temperature of the target to rise. The temperature rise of the target material directly affects its physical and chemical properties. Sufficient neutrons for treatment can be generated based on a large amount of charged particle beams, but the side effect of the charged particle beams is that the target generates a lot of heat, which causes damage to the target. However, BNCT usually requires a high current requirement for the charged particles, and the beam with a high beam current increases the risk of damage to the target. By evaluating the temperature rise of the target during bombardment, the selection and design of the target material can be optimized so as to ensure sufficient stability and service life under the action of high-energy charged particle beams.

Despite significant research efforts, current methodologies often rely on simple theoretical models or approximate calculations, which are then converted into two-dimensional formulas or empirical formulas. In this process, the two-dimensional formula causes a three-dimensional heat source to be compressed into a two-dimensional form, and the heat density is higher, but the input mode does not conform to the actual situation. The two-dimensional model only allows surface-level input, and the empirical formula suffers from incompleteness or limited applicability. In this case, interpolation is needed to expand the applicability range, yet interpolation introduces additional errors. Consequently, the calculation accuracy of the above method in the prior art is significantly insufficient, and the method fails to accurately reflect the actual thermal effect.

### SUMMARY OF THE INVENTION

The objective of the present invention is to provide a numerical simulation method and device for target temperature rise, a storage medium and a terminal, so as to solve the technical problem that it is difficult to evaluate the temperature rise of a target in the prior art.

In a first aspect, the present invention provides a numerical simulation method for target temperature rise, comprising:
obtaining a target finite element model, and obtaining a beam parameter of a charged particle beam;
obtaining corresponding charged particle spatial position coordinate distribution data based on the beam parameter;
performing, based on the charged particle spatial position coordinate distribution data, coordinate fitting on a charged particle distribution in three dimensions to obtain an expression of a corresponding charged particle distribution along a preset two-dimensional direction as a charged particle distribution feature corresponding to the beam parameter;
obtaining a simulation operation condition parameter, and simulating a process of the beam bombarding the target finite element model based on the simulation operation condition parameter and the charged particle distribution feature using finite element analysis to obtain a target surface temperature distribution after the target finite element model is bombarded; wherein
the simulation operation condition parameter is a parameter for characterizing a physical environment where the simulation is located, and the preset two-dimensional direction is a combination of any two directions in the three dimensions.

In one embodiment of the present invention, the preset two-dimensional direction is a two-dimensional direction capable of constituting a plane where a target surface is located.

In one embodiment of the present invention, the performing, based on the charged particle spatial position coordinate distribution data, coordinate fitting on a charged particle distribution in three dimensions to obtain an expression of a corresponding charged particle distribution along a preset two-dimensional direction as a charged particle distribution feature corresponding to the beam parameter includes:
inputting the charged particle spatial position coordinate distribution data into a preset fitter to obtain a charged particle distribution expression corresponding to the charged particle spatial position coordinate distribution data distributed along the preset two-dimensional direction.

In one embodiment of the present invention, the performing, based on the charged particle spatial position coordinate distribution data, coordinate fitting on a charged particle distribution in three dimensions to obtain an expression of a corresponding charged particle distribution along a preset two-dimensional direction as a charged particle distribution feature corresponding to the beam parameter includes:
obtaining a charged particle distribution correction model, wherein the charged particle distribution correction model is used to generate corresponding two-dimensional charged particle position coordinate distribution data along the preset two-dimensional direction based on the charged particle spatial position coordinate distribution data;
correcting the charged particle spatial position coordinate distribution data using the charged particle distribution correction model to obtain the corresponding two-dimensional charged particle position coordinate distribution data along the preset two-dimensional direction based on the charged particle spatial position coordinate distribution data;
inputting the two-dimensional charged particle position coordinate distribution data into a preset fitter to obtain a charged particle distribution expression corresponding to the charged particle spatial position coordinate distribution data distributed along the preset two-dimensional direction; wherein
input data for correcting the charged particle spatial position coordinate distribution data using the charged particle distribution correction model includes: the charged particle spatial position coordinate distribution data, beam spot distribution type data, and beam spot scanning type data.

In one embodiment of the present invention, the obtaining a simulation operation condition parameter, and simulating a process of the beam bombarding the target finite element model based on the simulation operation condition parameter and the charged particle distribution feature using finite element analysis to obtain a target surface temperature distribution after the target finite element model is bombarded includes:
generating a custom function for finite element analysis software based on the charged particle distribution feature, and setting a primary source term and a secondary source term in the custom function;
obtaining the simulation operation condition parameter, and setting a corresponding parameter in the finite element analysis software based on the simulation operation condition parameter to simulate an operation condition corresponding to the simulation operation condition parameter in the finite element analysis software;
simulating the process of the beam bombarding the target finite element model in the finite element analysis software based on the custom function to obtain the target surface temperature distribution after the target finite element model is bombarded; wherein
the primary source term is used to characterize energy of charged particles in the charged particle distribution feature, and the secondary source term is used to characterize a bombardment range generated in the target finite element model when the beam bombards the target finite element model.

In one embodiment of the present invention, before simulating the process of the beam bombarding the target finite element model in the finite element analysis software based on the custom function to obtain the target surface temperature distribution after the target finite element model is bombarded, further includes:
obtaining a target shooting parameter, and setting a corresponding source term in the custom function based on the target shooting parameter; wherein
the target shooting parameter includes a beam spot distribution type, and a source term corresponding to the beam spot distribution type is a distribution type source term.

In one embodiment of the present invention, the target shooting parameter further includes a beam spot scanning type, and the source term corresponding to the beam spot scanning type is a scanning type source term.

In a second aspect, the present invention further provides a numerical simulation device for target temperature rise, characterized by comprising:
a data obtaining module, configured to obtain a target finite element model and obtain a beam parameter of a charged particle beam;
a data acquisition module, configured to obtain corresponding charged particle spatial position coordinate distribution data based on the beam parameter;
a feature extraction module, configured to perform coordinate fitting on a charged particle distribution in three dimensions based on the charged particle spatial position coordinate distribution data to obtain an expression of the corresponding charged particle distribution along a preset two-dimensional direction as a charged particle distribution feature corresponding to the beam parameter;
a temperature simulation module, configured to obtain a simulation operation condition parameter, and simulate a process of the beam bombarding the target finite element model based on the simulation operation condition parameter and the charged particle distribution feature using finite element analysis to obtain a target surface temperature distribution after the target finite element model is bombarded; wherein
the simulation operation condition parameter is a parameter for characterizing a physical environment where the simulation is located, and the preset two-dimensional direction is a combination of any two directions in the three dimensions.

In a third aspect, the present invention further provides a storage medium storing computer programs, characterized in that when the computer programs are executed by a processor, the numerical simulation method for target temperature rise as described is implemented.

In a fourth aspect, the present invention further provides a terminal, comprising a processor and a memory, wherein the memory is in communication with the processor;
the memory is used to store computer programs, and the processor is used to execute the computer programs stored in the memory, so that the terminal executes the numerical simulation method for target temperature rise as described.

As described above, the numerical simulation method and device for target temperature rise, the storage medium and the terminal of the present invention have the following beneficial effects:

The present invention models the target and obtains the distribution of the charged particles, so as to convert the distribution of the charged particles in the charged particle beam to be bombarded into the heat distribution of the input target through fitting, and realize the numerical simulation evaluation of the temperature rise of the target based on the finite element method. Meanwhile, the present invention performs coordinate fitting on the charged particle distribution in three-dimensional directions, the obtained two-dimensional expression can better reflect the actual thermal effect, and the evaluation of the target temperature rise is more accurate.

### BRIEF DESCRIPTION OF DRAWINGS

FIG. 1 is a schematic diagram of BNCT treatment.

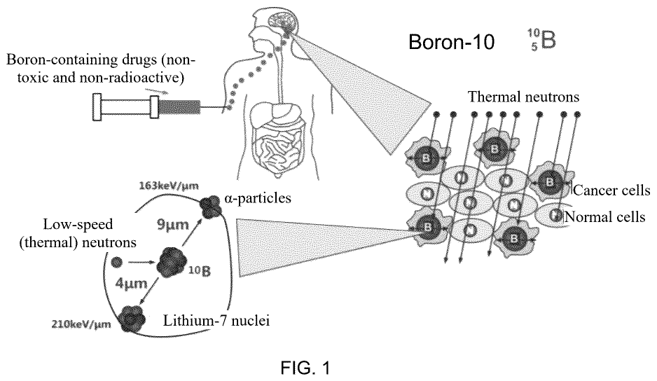

FIG. 2 is a schematic flowchart of a numerical simulation method for target temperature rise according to the embodiments of the present invention.

FIG. 3 is schematic diagrams of two target structures of a three-dimensional target model in a target finite element model according to the embodiments of the present invention.

FIG. 4 is a schematic diagram of two flowcharts for obtaining particle distribution expressions in a numerical simulation method for target temperature rise according to the embodiments of the present invention.

FIG. 5 is a schematic flowchart of simulating a process of a beam bombarding a target finite element model in a numerical simulation method for target temperature rise according to the embodiments of the present invention.

FIG. 6 is a schematic diagram of a temperature evaluation result of a numerical simulation method for target temperature rise according to the embodiments of the present invention.

FIG. 7 is a schematic structural diagram of a numerical simulation device for target temperature rise according to the embodiments of the present invention.

FIG. 8 is a schematic structural diagram of a terminal according to the embodiments of the present invention.

### REFERENCE NUMERALS

10 represents a substrate layer unit, 20 represents a lithium layer unit, 30 represents a base unit, and 40 represents a cooling microchannel unit.

### DETAILED DESCRIPTION OF THE INVENTION

The following describes the embodiments of the present invention by specific examples, and those skilled in the art can easily understand other advantages and effects of the present invention from the contents disclosed in this specification. The present invention can also be implemented or applied through other different specific embodiments, and the details in this specification can also be modified or changed in various ways based on different viewpoints and applications without departing from the spirit of the present invention. It should be noted that the following embodiments and features in the embodiments can be combined with each other without conflict.

The numerical simulation method and device for target temperature rise, the storage medium and the terminal of the present invention will be explained in detail below, and those skilled in the art can understand the disclosed numerical simulation method and device for target temperature rise, the storage medium and the terminal without creative work.

In order to solve the above technical problems existing in the prior art, the embodiments of the present invention provide a numerical simulation method for target temperature rise.

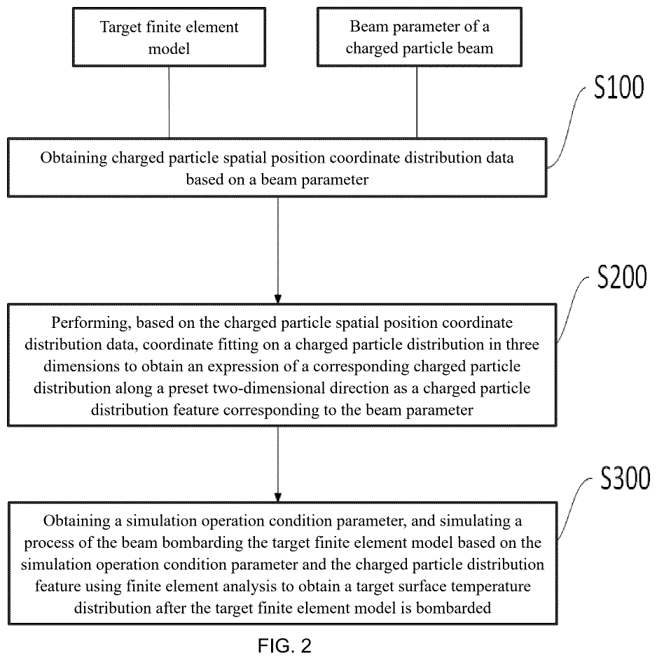

FIG. 2 is a schematic flowchart of a numerical simulation method for target temperature rise according to the embodiments of the present invention. Referring to FIG. 2, the numerical simulation method for target temperature rise according to the embodiments of the present invention mainly includes the following steps:

Step S100: obtaining charged particle spatial position coordinate distribution data based on a beam parameter.

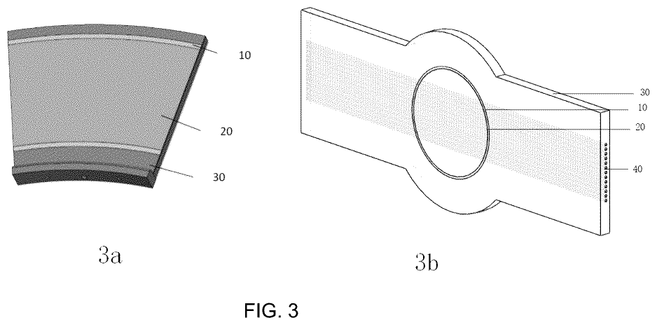

A target finite element model and the beam parameter of a charged particle beam are obtained as a data basis. Specifically, FIG. 3 is schematic diagrams of two target structures of a three-dimensional target model in a target finite element model according to the embodiments of the present invention. Referring to FIG. 3, Portion 3a is a partial structural diagram of a rotating target, and Portion 3b is a structural diagram of a fixed target. The two target structures are different, but are composed of the same components. In this embodiment, one of the above two target structures is selected to perform subsequent steps. As shown in FIG. 3, the three-dimensional target model includes a base unit 30, a substrate layer unit 10, a lithium layer unit 20 and a cooling microchannel unit 40, and the cooling microchannel unit 40 is distributed inside the base unit 30. The charged particle beam is composed of a large number of charged particles. The distribution of the charged particles in the beam has a correspondence with the beam parameter. The charged particle beam is used to bombard the target represented by the target finite element model to obtain the charged particle spatial position coordinate distribution data corresponding to the charged particle beam based on the beam parameter of the charged particle beam. The charged particle spatial position coordinate distribution data is capable of characterizing the charged particle beam to be bombarded on the target, and is a more specific data representation of the charged particle beam in a three-dimensional space. Simulating the target shooting process based on the data representation can more realistically simulate the temperature rise effect of the beam bombarding the target finite element model.

Optionally, the target finite element model is constructed in the following manner: establishing the three-dimensional target model, meshing the three-dimensional target model, i.e., meshing a fluid in the base unit 30, the substrate layer unit 10, the lithium layer unit 20, and the cooling microchannel unit, to obtain the three-dimensional target model after meshing as the target finite element model for subsequent simulation based on the finite element method. For example, the three-dimensional target model may be set as follows: a base material is copper, a base thickness is 10 mm; a substrate layer material is tantalum, a substrate layer thickness is 30 μm; the fluid in a cooling microchannel is water; a lithium layer thickness is 200 μm, and other target materials may be selected and different sizes may be set according to actual needs.

Furthermore, meshing the three-dimensional target model includes: dividing a base, the fluid, a substrate layer and a lithium layer using a structured mesh. The advantages of using the structured mesh for division include that it is time-saving and efficient, and the structured mesh has high calculation accuracy. Furthermore, after meshing, a mesh independence test is performed on a meshing result to improve the calculation accuracy of subsequent simulation.

Optionally, the mode of obtaining the corresponding charged particle spatial position coordinate distribution data based on the beam parameter includes: inputting the beam parameter into beam dynamics software to obtain the charged particle spatial position coordinate distribution data corresponding to the beam parameter.

Specifically, the beam parameter is pre-input parameters of beam, and the beam parameter is input into the beam dynamics software to simulate the process of beam change, and the coordinate distribution data of a spatial position corresponding to each charged particle is output. The beam parameter includes an initial beam distribution, a count of charged particles, and a magnetic and electric field configuration in an accelerator. Preferably, the beam dynamics calculation software is Tracewin, which can be used to simulate and analyze the transmission process of the charged particle beams in the accelerator, study the trajectory, beam shape and energy distribution of the charged particles, etc. It can accurately calculate the trajectory of the charged particles in an electromagnetic field, analyze the evolution of the beam, and provide high-precision dynamics calculation of the charged particles, which is suitable for a complex accelerator system.

According to actual needs, other methods can be selected to generate the charged particle spatial position coordinate distribution data corresponding to the beam parameter.

Step S200: performing, based on the charged particle spatial position coordinate distribution data, coordinate fitting on a charged particle distribution in three dimensions, to obtain an expression of a corresponding charged particle distribution along a preset two-dimensional direction as a charged particle distribution feature corresponding to the beam parameter.

It should be noted that the preset two-dimensional direction may be a combination of any two directions in the three-dimensional directions. For example, the three-dimensional spatial directions are x, y and z directions, and the preset two-dimensional direction may be x and y directions, x and z directions, or y and z directions. In the prior art, the charged particle distribution feature is obtained based on a simple theoretical model or approximate calculation, and then the calculation result is converted into an empirical formula. This method has significantly insufficient calculation accuracy. The present invention performs coordinate fitting on the charged particles in three dimensions, and can obtain the overall distribution of the charged particles in space based on a comprehensive perspective of the actual distribution of charged particles in space, so that further simulation is more consistent with the original distribution of the charged particles in the three-dimensional space.

Optionally, the preset two-dimensional direction is a combination of two-dimensional directions of a plane where the charged particles can form a target surface. Specifically, x, y and z representing the three-dimensional directions is taken as an example, y represents a target thickness direction of the charged particles, representing a main path of the beam entering and passing through the target. The x direction and the z direction are both perpendicular to the y direction. The x direction and the z direction form the two-dimensional plane where the target surface is located, i.e., a horizontal diffusion direction of the charged particles along the target surface. The x direction and the z direction reflect the distribution of the charged particles on the target surface, so as to understand the scattering or expansion of the charged particles on the target surface. Through the above process of fitting the charged particle spatial position coordinate distribution data in the three-dimensional directions to the distribution in the two-dimensional direction, the expression of the charged particle distribution in the x and z directions can be obtained, which is equivalent to reducing the dimensionality of the three-dimensional heat distribution in the target thickness direction, and compressing the original three-dimensionally distributed charged particles to the direction of the two-dimensional surface of the target. The dimensionality reduction compression along the y direction can directly reflect the temperature and heat distribution of the target surface (i.e., the plane formed by x and z directions), which is consistent with the actually measured and monitored physical surface. The expression of the charged particle distribution on the two-dimensional surface of the target formed by the x and z directions can represent the distribution rule of the charged particles in the target and can be used for further simulation analysis and calculation. Meanwhile, the expression of the two-dimensional distribution is also conducive to simplifying the calculation complexity.

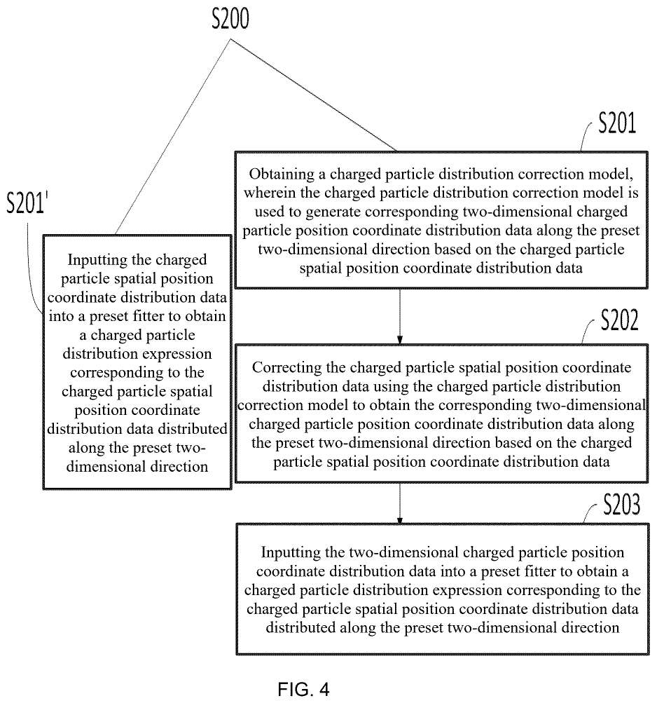

In the specific implementation of step S200, the present invention is implemented by a preset fitter. FIG. 4 is a schematic diagram of two flowcharts for obtaining particle distribution expressions in a numerical simulation method for target temperature rise according to the embodiments of the present invention. Referring to FIG. 4, coordinate fitting is performed on the charged particle distribution in three dimensions based on the charged particle spatial position coordinate distribution data, and the corresponding expression of the charged particle distribution in the preset two-dimensional direction can be obtained in two modes.

Mode 1: including step S201′, inputting the charged particle spatial position coordinate distribution data into a preset fitter to obtain a charged particle distribution expression corresponding to the charged particle spatial position coordinate distribution data distributed along the preset two-dimensional direction.

The preset fitter has the function of fitting the coordinate distribution data into an expression. Optionally, the preset fitter is a curve fitter in matlab. After the charged particle spatial position coordinate distribution data is imported into matlab, multiple Gaussian fittings are performed on the charged particle distribution based on the charged particle spatial position coordinate distribution data using the curve fitter in matlab, to obtain the expression of the charged particles along the preset two-dimensional direction. Optionally, the expression of the charged particles along the preset two-dimensional direction is:

$$f ( a ) = {\sum }_{i = 1}^{n} \frac{1}{\sqrt{2 π} {σ}_{i}} exp ( - \frac{{( a - {μ}_{i} )}^{2}}{2 {σ}_{i}^{2}} )$$

Where σ is a standard deviation of fitting, u is a mathematical expectation, and a is any direction in the preset two-dimensional direction. The specific expression corresponding to different beam spot distribution types can be obtained based on the above expression. Specifically, taking actual fitting data of the charged particle spatial position coordinate distribution data of a beam as an example for coordinate fitting when x and z are used as the preset two-dimensional directions, the charged particle spatial position coordinate distribution data is input into the preset fitter for fitting, and the following results can be obtained:

(1) If the beam spot presents a quasi-Gaussian distribution, the expression of the charged particles along the x and z directions is:

$$f ( x ) = 0.002186 * exp ( {( ( x 0.001671 ) / 0.01155 )}^{2} ) + 0.006651 * exp ( {( ( x 0.0001524 ) / 0.01682 )}^{2} )$$

$$f ( z ) = 0.005644 * exp ( - {( ( z - 0.001917 ) / 0.00757 )}^{2} ) + 0.00242 * exp ( - {( ( z - ( - 0.007224 ) ) / 0.005927 )}^{2} )$$

(2) If the beam spot presents a quasi-rectangular uniform distribution, the expression of the charged particles along the x and z directions is:

$$f ( x ) = 0.001429 * exp ( {( ( x + 0.04511 ) / 0.00247 )}^{2} ) + 0.01028 * exp ( {( ( x + 0.04147 ) / 0.005754 )}^{2} ) + 0.01529 * exp ( {( ( x + 0.04227 ) / 0.008133 )}^{2} )$$

$$f ( z ) = 0.0008898 * exp ( {( ( ( - z ) - 0.04991 ) / 0.0006292 )}^{2} ) + 0.0003926 * exp ( - {( ( ( - z ) - 0.04884 ) / 0.0005909 )}^{2} ) + 0.0002819 * exp ( - {( ( ( - z ) - 0.04902 ) / 0.00003756 )}^{2} ) + 0.005126 * exp ( - {( ( ( - z ) - 0.05053 ) / 0.003206 )}^{2} ) + 0.001648 * exp ( - {( ( ( - z ) - 0.04589 ) / 0.0008633 )}^{2} ) + 0.002455 * exp ( - {( ( ( - z ) - 0.04681 ) / 0.001847 )}^{2}$$

(3) If the beam spot presents a strip distribution, the expression of the charged particles along the x and z directions is:

$$f ( x ) = 0.0116 * exp ( - {( ( x - ( - 0.00365 ) ) / 0.002832 )}^{2} ) + 0.01635 * exp ( - {( ( x - 0.0007112 ) / 0.00495 )}^{2} )$$

$$f ( z ) = 0.005022 * exp ( - {( ( z - 0.01399 ) / 0.02866 )}^{2} ) + 0.004257 * exp ( - {( ( z - ( - 0.0279 ) ) / 0.02138 )}^{2} ) + 0.003082 * exp ( - {( ( z - 0.04427 ) / 0.0104 )}^{2} ) + 0.002894 * exp - {( ( z - ( - 0.04743 ) ) / 0.006885 )}^{2} )$$

The above fitting method can be applied to various distributions, such as a uniform circle, and the expression along the preset two-dimensional direction can also be other formulas, including but not limited to Gaussian formulas, polynomials, power functions, exponential functions, or a combination of multiple formulas. Accordingly, all distributions can be fitted using the preset fitter. This method uses a universal fitter, has a wider range of applications, and has a faster fitting speed.

Mode 2 comprises correcting the three-dimensional data by a preset model and then generating a two-dimensional expression using the preset fitter. As shown in FIG. 4, Mode 2 includes steps S201 to S203.

Step S201: obtaining a charged particle distribution correction model, wherein the charged particle distribution correction model is used to generate corresponding two-dimensional charged particle position coordinate distribution data along the preset two-dimensional direction based on the charged particle spatial position coordinate distribution data.

Specifically, the charged particle distribution correction model is obtained by training a preset model based on a charged particle distribution correction data set. The distribution correction data set includes multiple pieces of charged particle distribution correction training data, each of which uses the charged particle spatial position coordinate distribution data, the beam spot distribution type in the beam parameter, and the scanning type when the beam bombards the target finite element model as prediction factors, and uses the two-dimensional charged particle position coordinate distribution data corresponding to the charged particle spatial position coordinate distribution data as a prediction result. It should be noted that the preset model is selected based on the distribution features of the charged particles by assigning corresponding fitting function terms. Specifically, when the distribution of the charged particles is evidently similar to certain typical distribution patterns, the corresponding distribution function is adopted as the preset model. For example, if the distribution of charged particles exhibits a centrally high and peripherally low pattern on a two-dimensional plane, a normal function is selected as the preset model; whereas if the distribution appears uniform across the two-dimensional plane, a polynomial function is used as the preset model.

Step S202: correcting the charged particle spatial position coordinate distribution data using the charged particle distribution correction model, to obtain the corresponding two-dimensional charged particle position coordinate distribution data along the preset two-dimensional direction based on the charged particle spatial position coordinate distribution data.

Input data for correcting the charged particle spatial position coordinate distribution data using the charged particle distribution correction model includes: the charged particle spatial position coordinate distribution data, beam spot distribution type data, and beam spot scanning type data.

Step S203: inputting the two-dimensional charged particle position coordinate distribution data into a preset fitter to obtain a charged particle distribution expression corresponding to the charged particle spatial position coordinate distribution data distributed along the preset two-dimensional direction.

By correcting the charged particle spatial position coordinate distribution data through the charged particle distribution correction model, nonlinear features can be better extracted and reflected in the two-dimensional charged particle position coordinate distribution data, and the two-dimensional charged particle position coordinate distribution data is then input into the preset fitter to obtain the charged particle distribution expression. Compared with directly using the preset fitter, this processing mode can extract more complex and higher-order features from the original three-dimensional data through the charged particle distribution correction model. These features capture the complex patterns of the charged particle distribution and the potential relationships hidden in the data, making the output two-dimensional distribution data more accurate and representative. The two-dimensional distribution data is then input into the preset fitter for coordinate fitting, and the finally obtained charged particle distribution expression is more accurate, thereby improving the simulation effect.

Optionally, the preset model is an Extreme Gradient Boosting (XGBoost) model or a Convolutional Neural Network (CNN) model. The XGBoost model is known for its efficient computing power and good scalability. It can maintain efficient training and prediction performance when processing large-scale data sets. The XGBoost model can effectively capture nonlinear relationships in the data by using gradient boosting decision trees. When processing the charged particle distribution data, it can better model complex distribution features and quickly complete data correction tasks. It is suitable for processing large-scale charged particle spatial position coordinate distribution data. The CNN model is good at processing high-dimensional data, especially when processing three-dimensional charged particle spatial position coordinate distribution data. It can make full use of the spatial structure information of the data, thereby providing more accurate two-dimensional charged particle position coordinate distribution data than convolutional modes. Furthermore, in practical applications, other appropriate models can be selected according to specific data features and task requirements, or hybrid modeling can be performed by combining the advantages of the two models. For example, the XGBoost model is first used for preliminary correction, and then the CNN model is used for deep feature extraction and fine correction, so as to obtain more accurate and reliable charged particle distribution correction results.

Step S300: obtaining a simulation operation condition parameter, and simulating a process of the beam bombarding the target finite element model based on the simulation operation condition parameter and the charged particle distribution feature using finite element analysis, to obtain a target surface temperature distribution after the target finite element model is bombarded.

The simulation operation condition parameter is a parameter used to characterize a physical environment where the simulation is located, and the simulation operation condition parameter represents an actual operation condition. Optionally, the simulation operation condition parameter includes a preset solver model, a preset boundary condition, and a preset initial condition. Specifically, the simulation operation condition parameter is stored in a configuration file, and the simulation operation condition parameter is obtained by reading the simulation operation condition parameter from the configuration file. Those skilled in the art may also select other modes of obtaining the simulation operation condition parameter.

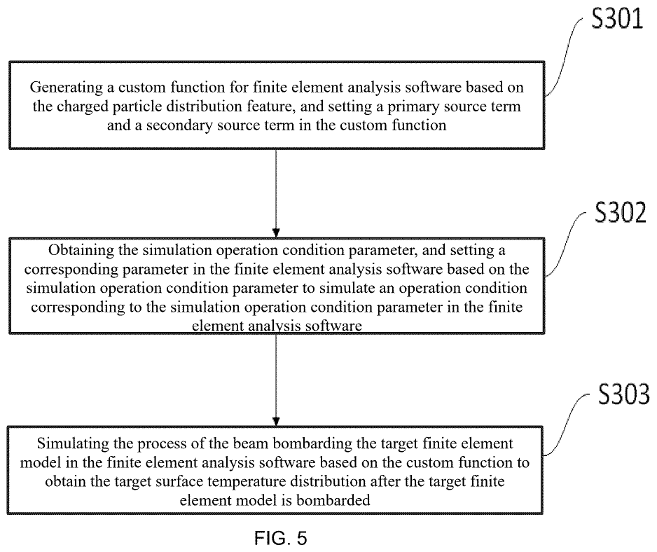

In one embodiment of the present invention, the above simulation process is performed using the finite element analysis software. FIG. 5 is a schematic flowchart of simulating a process of a beam bombarding a target finite element model in a numerical simulation method for target temperature rise according to the embodiments of the present invention. Referring to FIG. 5, the obtaining a simulation operation condition parameter, and simulating a process of the beam bombarding the target finite element model based on the simulation operation condition parameter and the charged particle distribution feature using finite element analysis to obtain a target surface temperature distribution after the target finite element model is bombarded includes the following steps:

Step S301: generating a custom function for finite element analysis software based on the charged particle distribution feature, and setting a primary source term and a secondary source term in the custom function.

Specifically, the finite element analysis software has a User-Defined Function (UDF) file for simulation calculation. UDFs that characterize the spatial distribution features of charged particles can be written in the UDF file. For example, charged particle distribution features obtained from Tracewin or other beam simulation software are typically presented as scatter plot data. These point-based scatter plots can be fitted into two-dimensional distribution functions using fitting software. If the depth distribution of the charged particles or their deposited energy within the target also needs to be considered, this distribution can likewise be fitted with a function. By combining it with the planar distribution function, a three-dimensional distribution function can be obtained. This three-dimensional function expression is then set as a UDF during the computational process. The primary source term and the secondary source term are set in the UDFs. The primary source term is used to characterize energy of the charged particles in the charged particle distribution feature, and the secondary source term is used to characterize a bombardment range generated in the target finite element model when the beam bombards the target finite element model. Specifically, the primary source term and the secondary source term are used as source terms of the input target surface. The primary source term is a source term q1 of the lithium layer, q1=Q*f(a), where Q represents a preset heat value, f(a) represents a charged particle expression of the charged particle distribution feature, q1 is a product of the preset heat value and the charged particle distribution feature, the preset heat value is determined by the energy and current of the charged particles, and q1 is used to characterize the energy of the charged particles in the charged particle distribution feature. The secondary source term is a source term q2 of the substrate layer, q2=V*f(a), where V represents a dynamic heat value, f(a) represents the charged particle expression of the charged particle distribution feature, q2 is a product of the dynamic heat value and the charged particle distribution feature, the dynamic heat value is a heat that changes along the depth, and the dynamic heat value is related to a range of the charged particles in the material, and q2 is used to characterize the bombardment range generated in the target finite element model when the beam bombards the target finite element model. Constructing the source term through the charged particle distribution feature and writing into the UDF file for simulation calculation can lay a data foundation for the subsequent accurate simulation of the beam bombarding the target finite element model, which is conducive to improving the simulation accuracy.

Furthermore, the finite element analysis software is Computational fluid dynamics (CFD) finite element analysis software. The CFD simulation software is a computational tool used to simulate and analyze fluid flow, heat transfer and related phenomena. It uses numerical modes and algorithms to solve fluid dynamics equations so as to simulate the behavior of the fluid under various conditions. In the CFD finite element analysis software, the process of the beam bombarding the finite element model is simulated based on the simulation parameter and the charged particle distribution feature, enabling the position distribution of the charged particles in the charged particle beam to be converted, through fitting, into the heat distribution of the input target, thereby effectively providing an important reference for the design of the target and the design of the high-energy beam line.

Preferably, the CFD finite element analysis software is Fluent, which is CFD software developed by ANSYS, providing multiple solvers and post-processing tools, supporting parallel computing, and capable of processing complex geometric structures and high-resolution models.

Step S302: obtaining the simulation operation condition parameter, and setting a corresponding parameter in the finite element analysis software based on the simulation operation condition parameter, to simulate an operation condition corresponding to the simulation operation condition parameter in the finite element analysis software.

Specifically, the simulation operation condition parameter is obtained, the corresponding parameter in the finite element analysis software is set according to the simulation operation condition parameter, the corresponding parameter is set to a parameter value of the simulation operation condition parameter, a solver model of the finite element analysis software is set to the preset solver model, a boundary condition of the finite element analysis software is set to the preset boundary condition, an initial condition of the finite element analysis software is set to the preset initial condition, and an operation condition is set for the numerical simulation process. Preferably, the solver model is a realizable k-ε model, and when performing simulation based on the model, it is not necessary to consider the selection of a boundary layer y+, and the accuracy is more accurate.

The simulation operation condition parameter is a parameter used to characterize the physical environment where the simulation is located, and the simulation operation condition parameter represents an actual operation condition. Optionally, the simulation operation condition parameter includes a preset solver model, a preset boundary condition, and a preset initial condition. The preset boundary condition includes physical environmental conditions under which the simulation is performed, such as temperature and radiation. As an example, regarding temperature, when the target is in a vacuum environment, the preset boundary condition is an adiabatic boundary condition, indicating that there is essentially no convective heat transfer due to the absence of gas. Conversely, when the target is fully or partially exposed to an atmospheric environment, the preset boundary condition is set as a convective boundary condition, indicating that the heat from the target disturbs the surrounding air and induces heat exchange. Similarly, a radiation boundary condition may also be set to represent heat dissipation from the target via radiation. The preset initial condition includes the initial state of the target during the simulation process. For instance, when the target begins the heating process, the preset initial condition may be set to a room temperature state to simulate the heating progression. Conversely, when the target begins the cooling process, the initial condition may be set to the state after thermal equilibrium to simulate the cooling behavior. Furthermore, the simulation operation condition parameter further includes a preset wall function, a preset cooling water flow rate, and a preset cooling water temperature. Optionally, the preset wall function is a standard wall function (SWF); the preset cooling water flow rate is 10 m/s, and the preset cooling water temperature is 283K. The wall function, the cooling water flow rate, and the cooling water temperature of the finite element analysis software are set accordingly based on the simulation operation condition parameter.

Step S303: simulating the process of the beam bombarding the target finite element model in the finite element analysis software based on the custom function, to obtain the target surface temperature distribution after the target finite element model is bombarded.

After the corresponding configuration of the simulation operation condition is completed in the finite element analysis software, the target shooting process of the beam bombarding the target finite element model can be simulated in the finite element analysis software, and a simulation result of the surface temperature distribution of the target finite element model after bombardment can be automatically generated and output.

It should be noted that, in other embodiments, those skilled in the art may also implement step S300 by using other existing finite element analysis modes such as equation calculation, data-driven and hybrid methods. For example, when using the equation calculation, the above setting as the custom function may be replaced by a shape function of the finite element analysis set in the equation calculation as an adaptive modification to the change in method. Those skilled in the art may implement the specific solution content based on the description of the present invention, which is not described in detail the present invention.

The primary source term and the secondary source term set in the custom function characterize the distribution feature of the charged particles. In order to improve the accuracy of the simulation, the present invention also considers setting other source terms. A target shooting parameter is a key parameter for controlling a beam feature and an interaction condition between the beam and the target. Optionally, before simulating the process of the beam bombarding the target finite element model in the finite element analysis software based on the custom function to obtain the target surface temperature distribution after the target finite element model is bombarded, it also includes: obtaining the target shooting parameter, and setting a corresponding source term in the custom function based on the target shooting parameter; wherein the target shooting parameter includes a beam spot distribution type, and a source term corresponding to the beam spot distribution type is a distribution type source term. Specifically, the beam spot distribution type is a quasi-Gaussian distribution, a quasi-rectangular uniform distribution, or a strip distribution. When writing the custom function, the target shooting parameter is a preset parameter set for the target shooting process. The beam spot distribution type represents the distribution of the beam in the preset two-dimensional direction of the target finite element model. By setting the source term corresponding to the beam spot distribution type in the UDF file based on the target shooting parameter, the beam distribution can be directly embedded as a source term in the simulation of the finite element analysis software, thereby more realistically reflecting the actual physical process. This approach can improve the accuracy of the simulation for considering the actual spatial distribution of the charged particles on the target.

The above process does not consider the movement of the beam spot, which is equivalent to simulating the target shooting with the beam spot in a fixed irradiation mode. In one embodiment of the invention, the movement of the beam spot is considered on the basis of the above process. Optionally, the target shooting parameter further includes the beam spot scanning type, and the source term corresponding to the beam spot scanning type is the scanning type source term. The beam spot scanning type is triangular wave scanning or reciprocating scanning irradiation. When the beam spot needs to move during target shooting, by setting the beam spot scanning type as a source term in the UDF file, the simulation process can consider the scanning mode of the charged particles, so that the simulation result can better reflect the actual temperature distribution.

Specifically, taking the movement when x and z are selected as the preset two-dimensional directions as an example, if the beam spot is set to scan in a triangular wave manner, the coordinates of each charged particle in the beam satisfy a movement trajectory along the x direction g(x)=(2*Ax/Tx)*(x % Tx)−Ax, and the movement trajectory along the z direction g(z)=(2*Az/Tz)*(z % Tz)−Az, where Ax represents a maximum scanning range of the charged particle in the x direction, Az represents a maximum scanning range of the charged particle in the z direction, Tx represents a period of the triangular waves of the charged particles in the x direction, Tz represents a period of the triangular waves of the charged particles in the z direction, and % is a remainder operation to ensure that the values of g(x) and g(z) cycle within one period; if the beam spot is set to reciprocate in one direction, then the coordinates of each charged particle in the beam only need to satisfy g(x)=(2*A/T)*(x % T)−A in that direction. According to different beam spot scanning types, the source terms of the corresponding beam spot scanning type can be set in the UDF file based on the above formula. Those skilled in the art can make corresponding settings for other preset two-dimensional modes based on the above description.

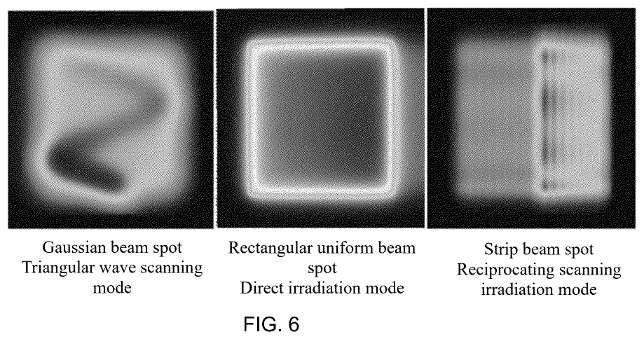

FIG. 6 is a schematic diagram of a temperature evaluation result of a numerical simulation method for target temperature rise according to the embodiments of the present invention. Referring to FIG. 6, the numerical simulation method for target temperature rise described in the embodiments of the present invention can obtain the temperature distribution of the target surface in a mode where beam spots of various distribution types irradiate the target with different beam spot scanning types. The beam deposition result is obtained through simulation, and the temperature rise features under different beam conditions are reflected. For example, the movement of the Gaussian beam spot have obvious movement traces, the uniform beam spot is a more regular model, and the strip beam spot has an obvious strip distribution.

The numerical simulation method for target temperature rise in the embodiments of the present invention is not limited to the execution order of the steps listed in this embodiment. All solutions implemented by adding, reducing or replacing steps in the prior art based on the principles of the present invention are included in the scope of the present invention.

The numerical simulation method for target temperature rise in the embodiments of the present invention models the target and obtains the distribution of the charged particles, so that the distribution of the charged particles in the charged particle beam to be bombarded is converted into the heat distribution of the input target through fitting in the finite element analysis software, thereby realizing the numerical simulation evaluation of the target temperature rise caused by charged particle shooting. Meanwhile, the coordinate fitting of the charged particle distribution is performed in the three-dimensional directions, and the obtained two-dimensional expression can better reflect the actual thermal effect, and the evaluation of the target temperature rise is more accurate. Therefore, it can effectively provide an important reference for the design of the target and the design of the high-energy beam line, and improve the treatment accuracy and safety of BNCT.

In order to solve the above technical problems existing in the prior art, the embodiments of the present invention further provide a numerical simulation device for target temperature rise.

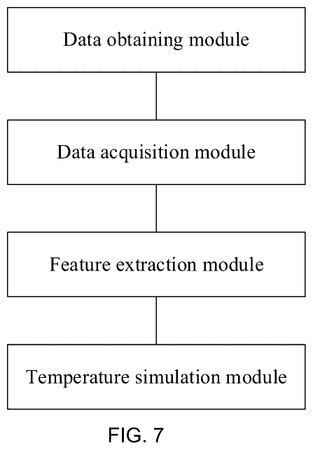

FIG. 7 is a schematic structural diagram of a numerical simulation device for target temperature rise according to the embodiments of the present invention. Referring to FIG. 7, the numerical simulation device for target temperature rise according to the embodiments of the present invention includes:
a data obtaining module, configured to obtain a target finite element model and obtain a beam parameter of a charged particle beam;
a data acquisition module, configured to obtain corresponding charged particle spatial position coordinate distribution data based on the beam parameter;
a feature extraction module, configured to perform coordinate fitting on a charged particle distribution in three dimensions based on the charged particle spatial position coordinate distribution data, to obtain an expression of the corresponding charged particle distribution along a preset two-dimensional direction as a charged particle distribution feature corresponding to the beam parameter;
a temperature simulation module, configured to obtain a simulation operation condition parameter, and simulate a process of the beam bombarding the target finite element model based on the simulation operation condition parameter and the charged particle distribution feature using finite element analysis, to obtain a target surface temperature distribution after the target finite element model is bombarded; wherein
the simulation operation condition parameter is a parameter for characterizing a physical environment where the simulation is located, and the preset two-dimensional direction is a combination of any two directions in the three dimensions.

As an example, the data obtaining module can incorporate a bus interface, DDR4 memory controller, Flash storage controller, and CRC-32 checksum circuit for efficient data retrieval. Similarly, the data acquisition module can utilize a RISC-V processor core, parallel multiply-accumulate unit, L2 cache, API interface controller, and UART to streamline data processing. Likewise, the feature extraction module can leverage a DSP core, Gaussian fitting accelerator, SRAM, and convolution computation unit to enhance analytical capabilities, while the temperature simulation module can integrate an ARM Cortex-A76, ARM Mali-G78, an embedded thermal conduction equation solver, and an SSD controller to support precise thermal modeling. The numerical simulation device for target temperature rise in the embodiments of the present invention models the target and obtains the distribution of the charged particles, thereby converting the distribution of the charged particles in the charged particle beam to be bombarded into the heat distribution of the input target through fitting in the finite element analysis software, and realizing the numerical simulation evaluation of the target temperature rise caused by charged particle shooting. Meanwhile, coordinate fitting is performed on the charged particle distribution in the three-dimensional directions, and the obtained two-dimensional expression can better reflect the actual thermal effect, and the evaluation of the target temperature rise is more accurate. Therefore, it can effectively provide an important reference for the design of the target and the design of the high-energy beam line, and improve the treatment accuracy and safety of BNCT.

In order to solve the above technical problems existing in the prior art, the embodiments of the present invention further provide a storage medium storing computer programs. When the computer programs are executed by a processor, all steps of the numerical simulation method for target temperature rise of the embodiments are implemented.

The specific steps of the numerical simulation method for target temperature rise and the beneficial effects obtained by applying the readable storage medium provided by the embodiments of the present invention are the same as those in the above embodiment, which are not described in detail herein.

It is understood for those having ordinary skills in the art that all or part of the steps in the method for implementing the above embodiments can be completed by instructing the processor through the programs, and the programs can be stored in the computer-readable storage medium. The storage medium is a non-transitory medium, such as a random-access memory, a read-only memory, a flash memory, a hard disk, a solid-state drive, a magnetic tape, a floppy disk, an optical disc, or any combination thereof. The storage medium may be any available medium that can be accessed by a computer or a data storage device such as a server or a data center that includes one or more available media. The available medium may be a magnetic medium (e.g., a floppy disk, a hard disk, and a magnetic tape), an optical medium (e.g., a digital video disc (DVD)), or a semiconductor medium (e.g., a solid state disk (SSD)).

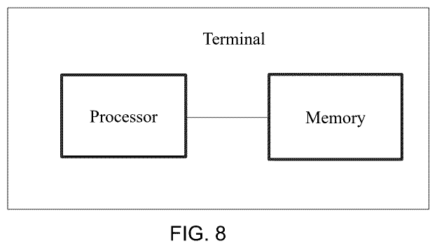

In order to solve the above technical problems existing in the prior art, the embodiments of the present invention further provide a terminal. FIG. 8 is a schematic structural diagram of a terminal according to the embodiments of the present invention. Referring to FIG. 8, the terminal of the embodiments of the present invention includes a processor and a memory, the memory and the processor being in communication. The memory is used to store computer programs, and the processor is used to execute the computer programs stored in the memory, so that the terminal executes all steps of the numerical simulation method for target temperature rise in the above embodiments.

The specific steps of the numerical simulation method for target temperature rise and the beneficial effects obtained by applying the terminal provided by the embodiments of the present invention are the same as those in the above embodiments, which are not described in detail herein.

It should be noted that the memory may include a random access memory (RAM) and may also include non-volatile memory, such as at least one disk storage. Similarly, the processor may also be a general-purpose processor, including a central processing unit (CPU), a network processor (NP), etc.; it may also be a digital signal processor (DSP), an application specific integrated circuit (ASIC), a field programmable gate array (FPGA), or other programmable logic devices, discrete gates or transistor logic devices, discrete hardware components.

Although the embodiments disclosed in the present invention are as above, the contents described are only embodiments adopted for facilitating the understanding of the present invention and are not intended to limit the present invention. Any person skilled in the art of the present invention belongs can make any modifications and changes in the form and details of the implementation without departing from the spirit and scope disclosed in the present invention. However, the scope of the present invention shall still be subjected to the protection scope defined in the attached claims.

1. A numerical simulation method for target temperature rise, comprising:
obtaining a target finite element model, and obtaining a beam parameter of a charged particle beam;
obtaining corresponding charged particle spatial position coordinate distribution data based on the beam parameter;
performing, based on the charged particle spatial position coordinate distribution data, coordinate fitting on a charged particle distribution in three dimensions, to obtain an expression of a corresponding charged particle distribution along a preset two-dimensional direction as a charged particle distribution feature corresponding to the beam parameter;
obtaining a simulation operation condition parameter, and simulating a process of the beam bombarding the target finite element model based on the simulation operation condition parameter and the charged particle distribution feature using finite element analysis, to obtain a target surface temperature distribution after the target finite element model is bombarded; wherein
the simulation operation condition parameter is a parameter for characterizing a physical environment where the simulation is located, and the preset two-dimensional direction is a combination of any two directions in the three dimensions.

2. The numerical simulation method according to claim 1, characterized in that the preset two-dimensional direction is a two-dimensional direction capable of constituting a plane where a target surface is located.

3. The numerical simulation method according to claim 1, characterized in that the performing, based on the charged particle spatial position coordinate distribution data, coordinate fitting on a charged particle distribution in three dimensions to obtain an expression of a corresponding charged particle distribution along a preset two-dimensional direction includes:
inputting the charged particle spatial position coordinate distribution data into a preset fitter, to obtain a charged particle distribution expression corresponding to the charged particle spatial position coordinate distribution data distributed along the preset two-dimensional direction.

4. The numerical simulation method according to claim 1, characterized in that the performing, based on the charged particle spatial position coordinate distribution data, coordinate fitting on a charged particle distribution in three dimensions to obtain an expression of a corresponding charged particle distribution along a preset two-dimensional direction includes:
obtaining a charged particle distribution correction model, wherein the charged particle distribution correction model is used to generate corresponding two-dimensional charged particle position coordinate distribution data along the preset two-dimensional direction based on the charged particle spatial position coordinate distribution data;
correcting the charged particle spatial position coordinate distribution data using the charged particle distribution correction model to obtain the corresponding two-dimensional charged particle position coordinate distribution data along the preset two-dimensional direction based on the charged particle spatial position coordinate distribution data;
inputting the two-dimensional charged particle position coordinate distribution data into a preset fitter, to obtain the charged particle distribution expression corresponding to the charged particle spatial position coordinate distribution data distributed along the preset two-dimensional direction; wherein
input data for correcting the charged particle spatial position coordinate distribution data using the charged particle distribution correction model includes: the charged particle spatial position coordinate distribution data, beam spot distribution type data, and beam spot scanning type data.

5. The numerical simulation method according to claim 1, characterized in that the obtaining a simulation operation condition parameter, and simulating a process of the beam bombarding the target finite element model based on the simulation operation condition parameter and the charged particle distribution feature using finite element analysis to obtain a target surface temperature distribution after the target finite element model is bombarded includes:
generating a custom function for finite element analysis software based on the charged particle distribution feature, and setting a primary source term and a secondary source term in the custom function;
obtaining the simulation operation condition parameter, and setting a corresponding parameter in the finite element analysis software based on the simulation operation condition parameter, to simulate an operation condition corresponding to the simulation operation condition parameter in the finite element analysis software;
simulating the process of the beam bombarding the target finite element model in the finite element analysis software based on the custom function, to obtain the target surface temperature distribution after the target finite element model is bombarded; wherein
the primary source term is used to characterize energy of charged particles in the charged particle distribution feature, and the secondary source term is used to characterize a bombardment range generated in the target finite element model when the beam bombards the target finite element model.

6. The numerical simulation method according to claim 5, characterized in that before simulating the process of the beam bombarding the target finite element model in the finite element analysis software based on the custom function to obtain the target surface temperature distribution after the target finite element model is bombarded, the numerical simulation method further includes:
obtaining a target shooting parameter, and setting a corresponding source term in the custom function based on the target shooting parameter; wherein
the target shooting parameter includes a beam spot distribution type, and a source term corresponding to the beam spot distribution type is a distribution type source term.

7. The numerical simulation method according to claim 6, characterized in that the target shooting parameter further includes a beam spot scanning type, and the source term corresponding to the beam spot scanning type is a scanning type source term.

8. A numerical simulation device for target temperature rise, characterized by comprising:
a data obtaining module, configured to obtain a target finite element model and obtain a beam parameter of a charged particle beam;
a data acquisition module, configured to obtain corresponding charged particle spatial position coordinate distribution data based on the beam parameter;
a feature extraction module, configured to perform coordinate fitting on a charged particle distribution in three dimensions based on the charged particle spatial position coordinate distribution data, to obtain an expression of the corresponding charged particle distribution along a preset two-dimensional direction as a charged particle distribution feature corresponding to the beam parameter;
a temperature simulation module, configured to obtain a simulation operation condition parameter, and simulate a process of the beam bombarding the target finite element model based on the simulation operation condition parameter and the charged particle distribution feature using finite element analysis, to obtain a target surface temperature distribution after the target finite element model is bombarded; wherein
the simulation operation condition parameter is a parameter for characterizing a physical environment where the simulation is located, and the preset two-dimensional direction is a combination of any two directions in the three dimensions.

9. A storage medium storing computer programs, characterized in that when the computer programs are executed by a processor, the numerical simulation method for target temperature rise as described in claim 1 is implemented.

10. A terminal, characterized by comprising a processor and a memory, wherein the memory is in communication with the processor; the memory is used to store computer programs, and the processor is used to execute the computer programs stored in the memory, so that the terminal executes the numerical simulation method for target temperature rise as described in claim 1.
